(19) United States
(12) Patent Application Publication
(10) Pub. No.: US 2022/0125543 A1
(43) Pub. Date: Apr. 28, 2022

(54) SURGICAL INSTRUMENT TRAY

(71) Applicant: DEPUY IRELAND UNLIMITED COMPANY, RINGASKIDDY (IE)

(72) Inventors: ALEC BIRKBECK, LEEDS - YORKSHIRE (GB); SARAH BUSHELL, LEEDS - YORKSHIRE (GB); DUNCAN YOUNG, LEEDS - YORKSHIRE (GB)

(21) Appl. No.: 17/284,827

(22) PCT Filed: Oct. 4, 2019
(86) PCT No.: PCT/EP2019/076981

§ 371 Date: Apr. 13, 2021

(30) Foreign Application Priority Data
Oct. 19, 2018 (GB) 1817051.4

(51) Int. Cl. A61B 50/33

(52) U.S. Cl. CPC A61B 50/33; A61B 2050/3006

(57) ABSTRACT

Surgical instrument trays and related methods are described. The surgical instrument tray is for a surgical instrument having a plurality of parts from which the surgical instrument is assembled. The surgical tray comprises a tray having a plurality of side walls and a base which define an interior space; a lid for closing the interior space and which is removable from the tray; and a plurality of outlines located within the interior space. Each outline has the shape of a corresponding different part of the surgical instrument and each outline has at least one support arranged to receive and releasably hold the respective different parts of the surgical instrument above and in registration with the outline. The plurality of outlines are configured to indicate that the different parts of the surgical instrument are assembled to form the surgical instrument and/or that the surgical instrument is disassembled into the different parts.

The present invention relates to trays and in particular surgical instrument trays for holding surgical instrument for use during a surgical procedure.

Surgical instrument trays are known generally and are typically used to store and transport the surgical instruments and any other tools accessories and parts that may be used in a surgical procedure. Surgical instrument trays typically have to meet a number of competing aims.

They need to be strong and robust in order to be able to protect and handle the surgical instruments, some of which may be quite heavy and/or delicate, during handling of the tray.

They should also be reasonably light so that they can be handled by various people, such as operating theatre staff and washroom staff.

They should be easily cleanable so that the trays can be easily and reliably sterilised for re-use.

They should be reasonably large so that a reasonable number of instruments can be stored in them and so that relatively few are actually needed in the operating theatre for any particular procedure, but not too large as to be too difficult to handle.

They should also be relatively simply in construction so as to be low cost and/or easy to manufacture.

They should also be user friendly so that it is easy for users to correctly load the surgical instruments into the tray and/or also to find and remove the correct surgical instrument from the tray when needed during surgery.

Hence, surgical instrument trays which help address one or more of these issues, and/or other issues with surgical instrument trays, may be beneficial.

According to a first aspect of the invention there is provided a surgical instrument tray for a surgical instrument having a plurality of parts from which the surgical instrument is assembled, the surgical tray comprising: a tray having a plurality of side walls and a base which define an interior space; and a plurality of outlines located within the interior space, each outline having the shape of a corresponding different part of the surgical instrument and wherein each outline has at least one support arranged to receive and releasably hold the respective different parts of the surgical instrument above and in registration with the outline and wherein the plurality of outlines are configured to indicate that the different parts of the surgical instrument are assembled to form the surgical instrument and/or that the surgical instrument is disassembled into the different parts.

The surgical instrument tray may further comprise a lid for closing the interior space and which is removable from the tray.

The plurality of outlines may be further configured to indicate how the different parts of the surgical instrument are assembled to form the surgical instrument and/or how the surgical instrument is disassembled into the different parts.

The plurality of outlines may be further configured to indicate the sequence in which the different parts of the surgical instrument are assembled to form the surgical instrument and/the sequence in which the surgical instrument is disassembled into the different parts.

The plurality of outlines may be configured by being grouped together.

The plurality of outlines may be configured by the relative positions of the plurality of outlines.

The plurality of outlines may be configured by colour coding of the plurality of outlines. The plurality of outlines may be the same colour. The plurality of outlines may be different shades or hues of the same colour.

The plurality of outlines may be configured by respective indicia associated with each outline.

Each indicium may be a different alpha and/or numeric character. The different alpha and/or numeric characters may indicate how the parts are assembled or disassembled and/or the sequence.

The plurality of outlines may be connected by respective lands. Each land may include an indicium or indicia that indicates how the parts are assembled or disassembled and/or the sequence.

The surgical instrument tray may further comprising a further plurality of outlines. Each of the further plurality of outlines may have the shape of a corresponding further surgical instrument or part of a further surgical instrument. Each outline may have at least one support arranged to receive and releasably hold the further surgical instrument or part of the further surgical instrument above and in registration with the outline.

Each outline may have an outer perimeter and the dimensions of the outer perimeter of the outline may be greater than the dimensions of the corresponding portions of the different parts of the surgical instrument, so that the outer perimeter of the outline is visible when the parts of the surgical instrument are located within the respective supports above the corresponding outlines.

The plurality of outlines may be provided separate to the base of the tray and/or as part of a sheet of material.

The sheet of material may be positioned above and spaced away from the base of the tray.

Each outline may be in the form of a strip, and preferably a continuous strip.

Each strip of material of each outline may define at least one aperture or void within the outline.

Each strip may have a width in the range of approximately 2 mm to 8 mm, preferably approximately 3 mm to 6 mm, and more preferably approximately 4 mm to 5 mm.

Each of the plurality of outlines may have an outline colour and the remainder of the surrounding material may have a surround colour. The outline colour and the surround colour may be different colours and/or may be contrasting colours. The outline colour may be white and the surround colour may be black. The outline colour may be black and the surround colour may be white.

The plurality of outlines may be made from a sheet of metal. The plurality of outlines may be have been created by cutting the sheet of metal.

Each support may be made of a resilient material. The resilient material may be a non-metallic material. The resilient material may be a synthetic rubber or polymer or similar. The resilient material may be silicone.

Each support may be in the form of a clip.

The surgical instrument tray may further comprise the plurality of parts from which the surgical instrument is assembled and the plurality of parts may be received in and held by the respective supports above and in registration with the corresponding respective outlines. The surgical instrument may be an orthopaedic surgical instrument.

A second aspect of the invention provides a method of using a surgical instrument tray to assemble a surgical instrument form a plurality of parts of the surgical instrument, wherein the surgical instrument tray comprises a tray having a plurality of side walls and a base which define an interior space and a plurality of outlines located within the interior space, each outline having the shape of a corresponding different part of the surgical instrument and wherein each outline has at least one support receiving and releasably holding the respective different parts of the surgical instrument above and in registration with the outline and wherein the plurality of outlines are configured to indicate that the different parts of the surgical instrument are assembled to form the surgical instrument, the method comprising; viewing the plurality of outlines to identify the parts of the surgical instrument to be assembled to form the surgical instrument; removing the identified parts of the surgical instrument from the supports; and assembling the identified parts of the surgical instrument to form the surgical instrument.

The plurality of outlines may be further configured to indicate the sequence in which the different parts of the surgical instrument are assembled to form the surgical instrument, and the method may comprise removing the identified parts and assembling the identified parts according to the sequence.

A third aspect of the invention provides a method of using a surgical instrument tray to disassemble a surgical instrument formed from a plurality of parts, wherein the surgical instrument tray comprises a tray having a plurality of side walls and a base which define an interior space and a plurality of outlines located within the interior space, each outline having the shape of a corresponding different part of the surgical instrument and wherein each outline has at least one support arranged to receive and releasably hold the respective different parts of the surgical instrument above and in registration with the outline and wherein the plurality of outlines are configured to indicate that the different parts of the surgical instrument are assembled to form the surgical instrument, the method comprising; disassembling at least one part of the surgical instrument; viewing the plurality of outlines to identify the support for the at least one part of the surgical instrument that has been disassembled; and placing the at least one disassembled part in the identified support above and in registration with the corresponding outline for the part.

The method may further comprise: disassembling all the parts of the surgical instrument; viewing the plurality of outlines to identify the respective supports for all the parts of the surgical instrument that have been disassembled; and placing all the parts in the respective identified supports above and in registration with the corresponding outlines for the parts.

The method of the second aspect of the invention and/or the method of the third aspect of the invention may be an automated method or a semi-automated method.

The preferred features of the first aspect of the invention may also give rise to preferred counterpart features of the second aspect of the invention and/or the third aspect of the invention.

Embodiments of the invention will not be described in detail, and by way of example only, with reference to the accompanying drawings, in which:

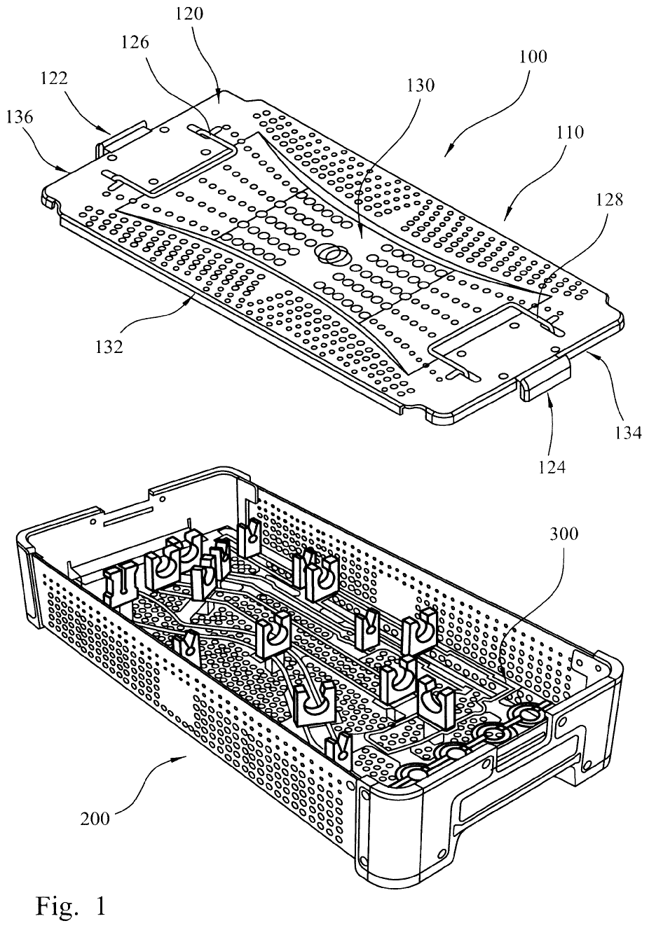

FIG. 1 shows a perspective view of a surgical instrument tray according to an embodiment of the invention;

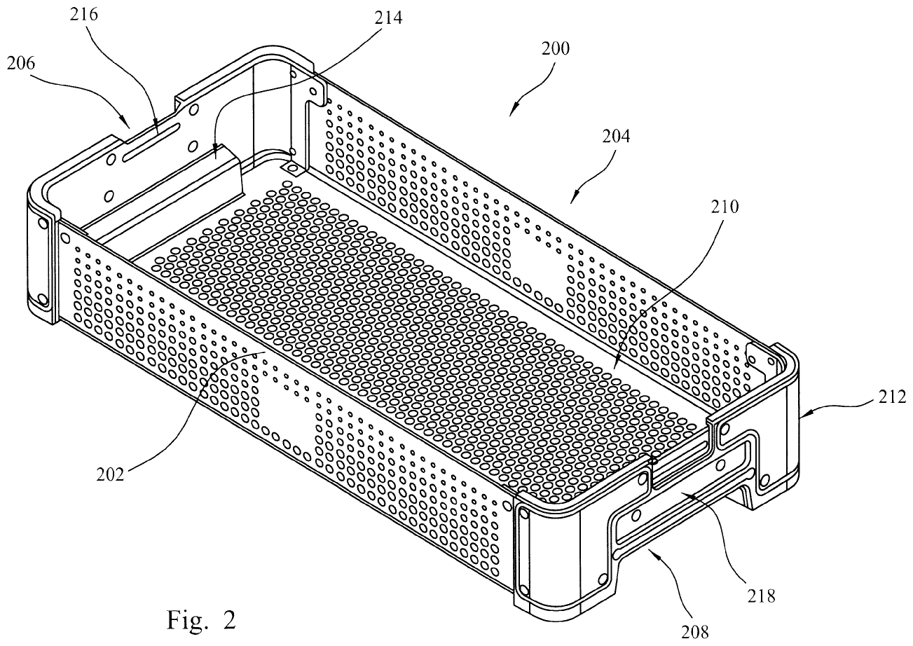

FIG. 2 shows a perspective view of a tray part of the surgical instrument tray of the invention;

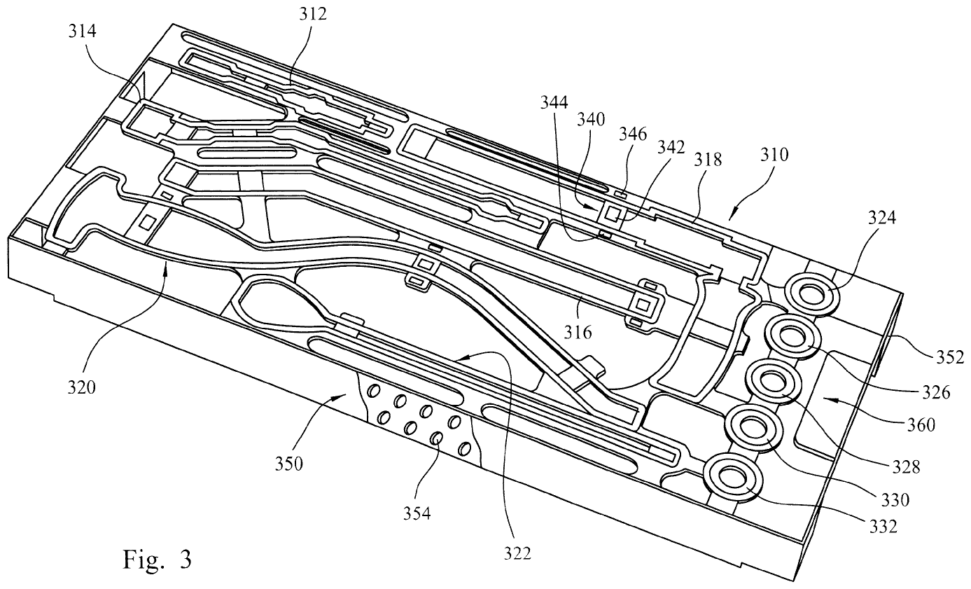

FIG. 3 shows a perspective view of a first embodiment of an outlines part of the surgical instrument tray of the invention;

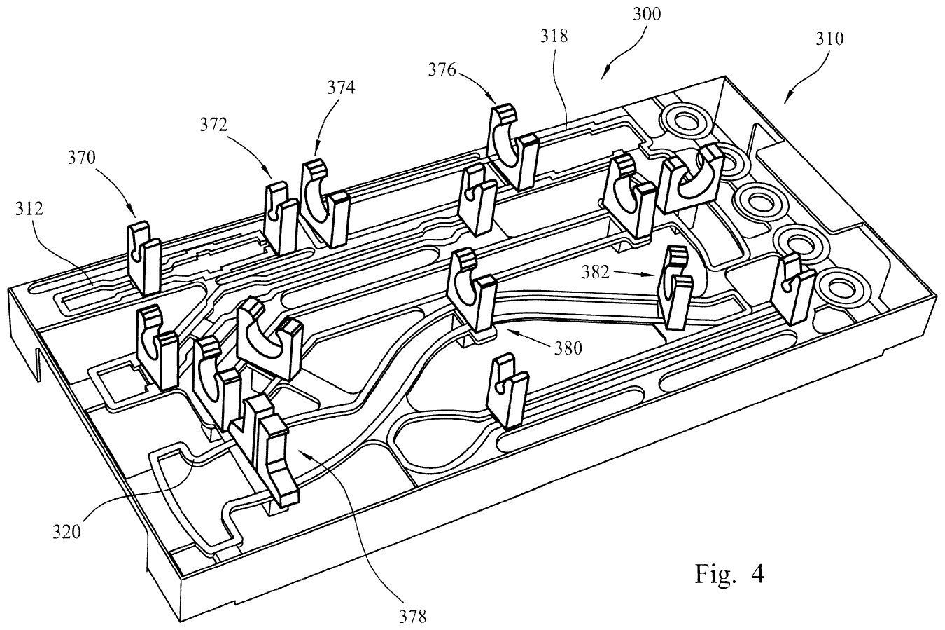

FIG. 4 shows a perspective view of an insert including the outlines of FIG. 3 and a plurality of supports;

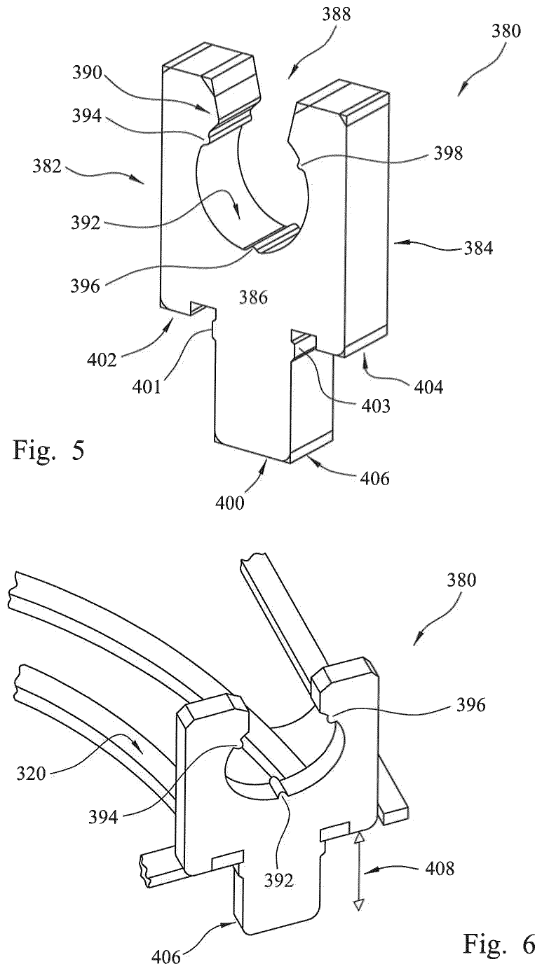

FIG. 5 shows a perspective view of a one of the supports illustrated in FIG. 4;

FIG. 6 shows a perspective view of a section through a one of the supports and a part of the outlines illustrating attachment of the support to the outline;

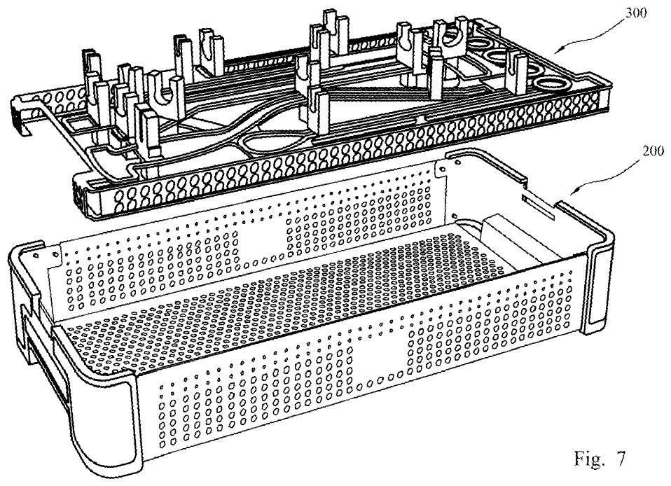

FIG. 7 shows a perspective view of the insert and tray in a separated configuration;

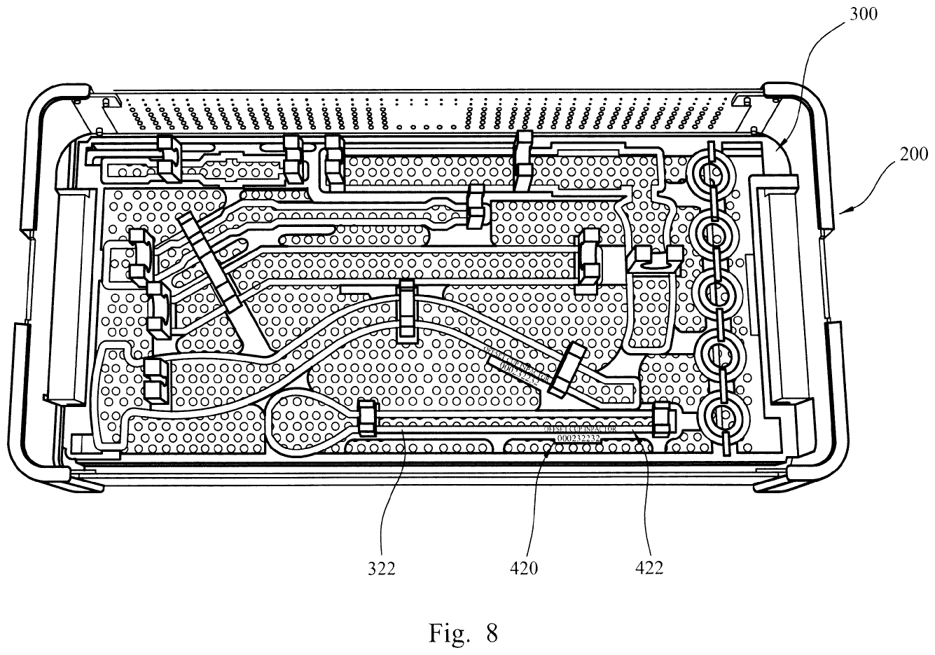

FIG. 8 shows a perspective view from above of the insert located within an interior space of the tray;

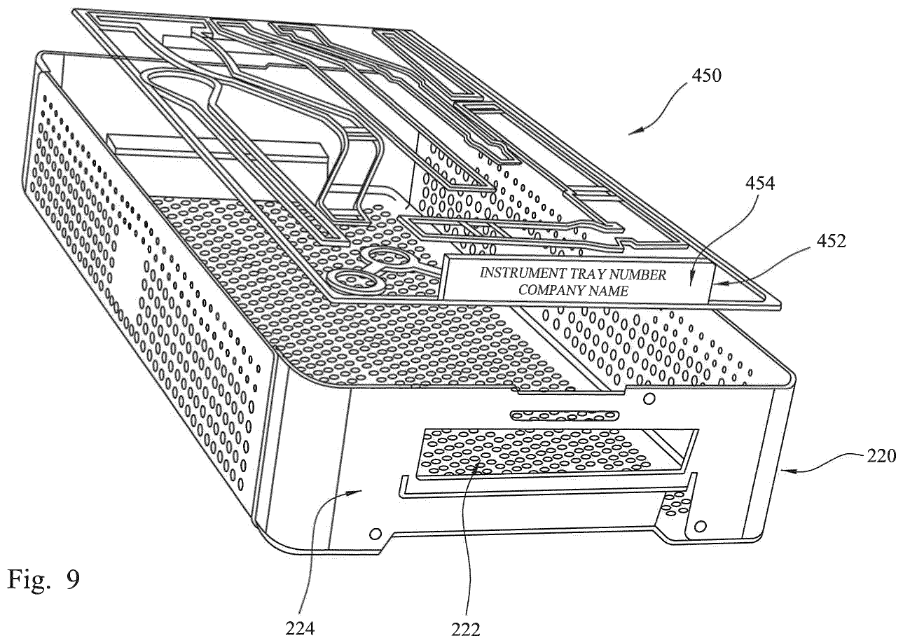

FIG. 9 shows a perspective view of a second embodiment of an insert and a tray;

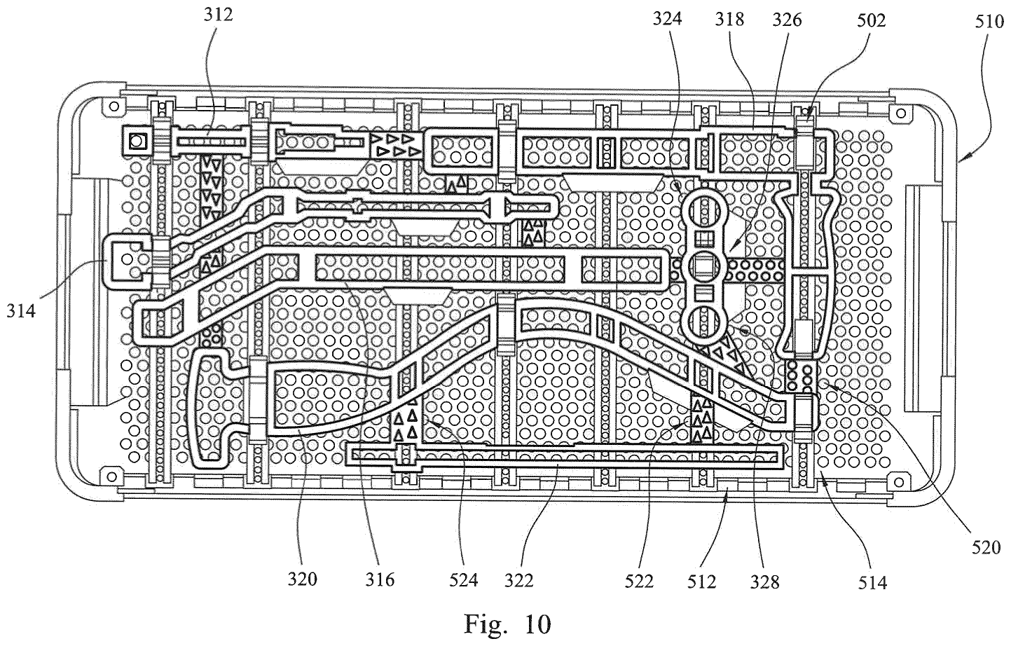

FIG. 10 shows a plan view of a third embodiment of the insert;

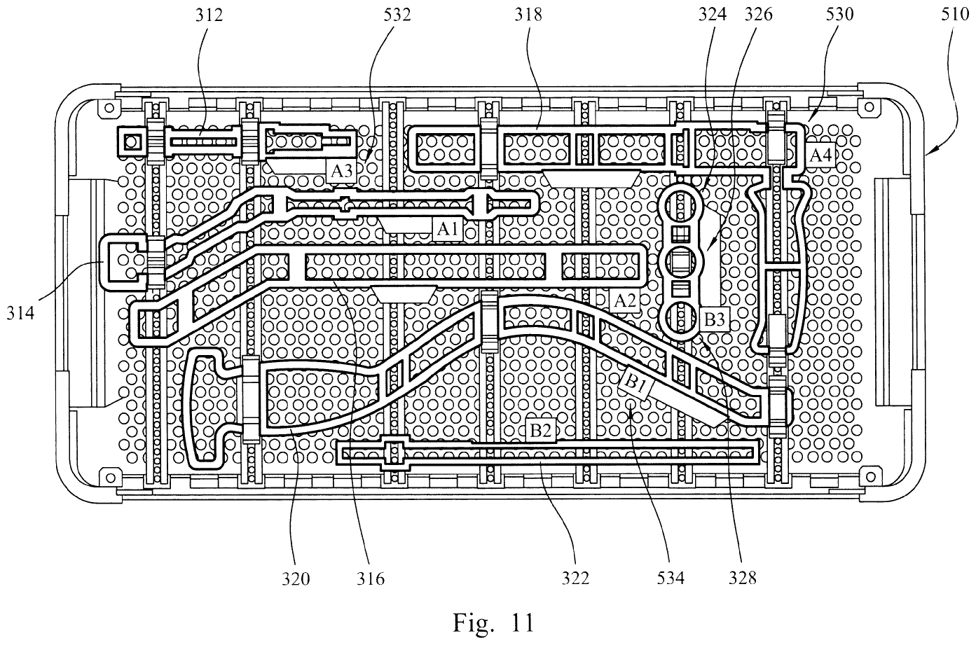

FIG. 11 shows a plan view of a fourth embodiment of the insert;

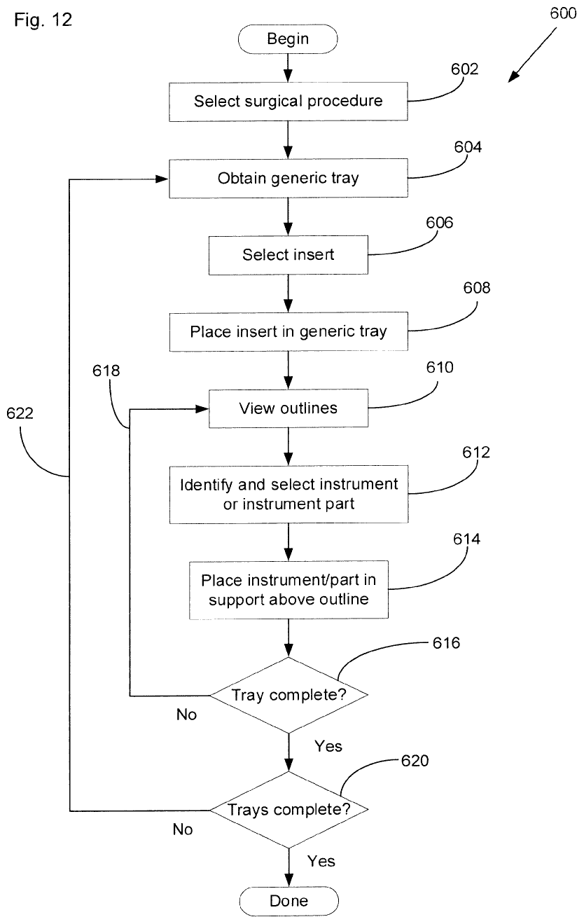

FIG. 12 shows a flow chart illustrating a method of producing surgical instrument trays according to an aspect of the invention; and

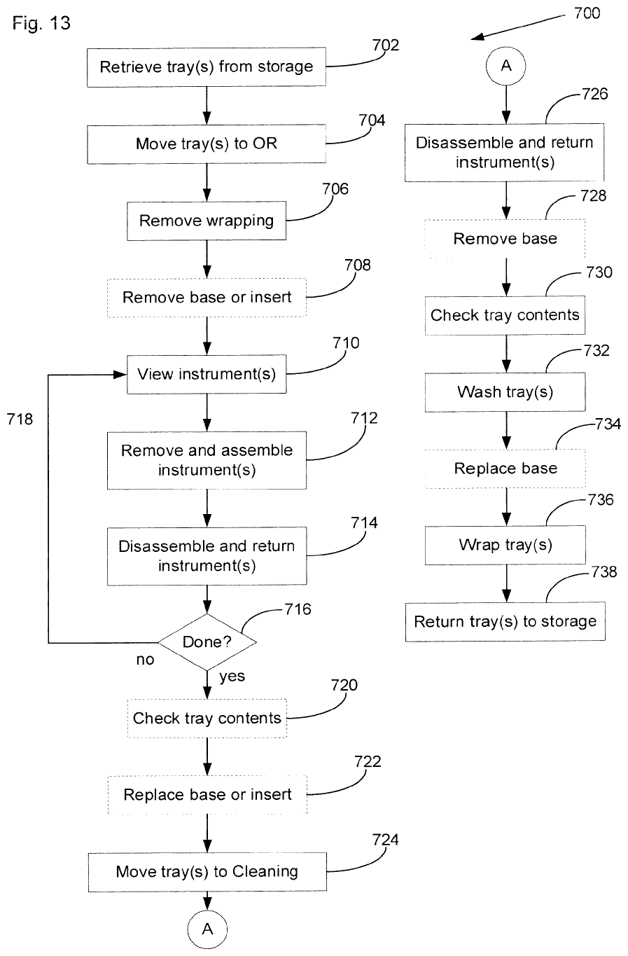

FIG. 13 shows a flow chart illustrating various methods of use of the surgical instrument tray according to various aspects of the invention.

In the Figures, similar items in different Figure share common reference numerals unless indicated otherwise.

The embodiments of the invention will be described within the context of orthopaedic surgery, but it will be appreciated that the invention is not limited in application to orthopaedic surgery. The invention may be used in a wide variety of fields of surgery in which various different surgical instruments and/or multi-part surgical instruments may be used.

With reference to FIG. 1 there is shown a perspective view of a surgical instrument tray according to an aspect of the invention. The surgical instrument tray generally includes a lid 110, a tray 200 and an insert 300 located in use within an anterior space defined by the tray 200. The lid 110 is removable from the tray and when attached helps to retain the tray contents within the surgical instrument tray. As explained in greater detail below, the contents of the surgical instrument tray may be individual surgical instruments, parts of surgical instruments which are assembled to form the actual surgical instrument and various other parts, accessories, tools and devices typically used during surgical procedures. In orthopaedic surgical procedures, the contents of the tray may also include trial implant components.

The lid 110 has a main body 112 made of a metal material and has a plurality of apertures therein to allow the ingress of water or steam during washing and sterilisation processes used to clean the surgical instrument tray. First 122 and second 124 latches are provided at opposed ends of the lid to allow removable attachment of the lid to the tray. First 126 and second 128 handles are also provided at opposed ends of the lid. Each handle may be pivotably attached to the body of the lid and may be made of a metal, such as stainless steel or aluminium, and may be foldable between a storage configuration, as illustrated in FIG. 1, and a carrying configuration in which they stand upright relative to the plain of the body 120. A central portion 130 of the body 120 may be configured to define a slight concavity relative to the flanks of the body 120 to enable a user to grasp the handles.

A short side wall, e.g. side wall 132, extends along the length of either side of the lid and is arranged, in use, to be located against an outer side of the tray side wall. The end 134, of the lid body 120 are configured to be received within the corresponding end portions of the mouth of the tray so that, when attached to the tray, the lid presents a substantially flush surface for the overall surgical instrument tray. This can aid stacking, storage and transportation of the surgical instrument trays in use.

FIG. 2 shows a perspective view of the tray 200 of the overall surgical instrument tray 100. Generally, herein, surgical instrument tray will be used to refer to the entire assembly 100 and tray will be used to refer to the tray part 200 shown in FIG. 2.

The tray 200 includes first 202 and second 204 side walls and first 206 and second 208 end walls. The tray also includes a base 210. The side walls, end walls and base define an interior space in which an insert may be removably received as described in greater detail below.

The side walls and end walls and base are made from a suitable metal, such as aluminium or stainless steel. The side walls, end walls and base may be joined together by riveting. The end walls may also include plastic and/or rubber curved, bumper-like parts to protect the tray and also avoid damage. These parts may also provide a cool surface to allow handling of the tray soon after sterilization. Each end wall has a shoulder, e.g. 214, toward a lower part extending into the interior space. The recesses toward the exterior formed by the shoulders provides a space to accommodate a user's hands to facilitate handling of the tray. An upper part of each end wall defines a slot 216 for receiving a part of the lid clips to allow the lid to be securely attached to the tray.

As illustrated in FIG. 2, the side walls and base include a large number of perforations to allow the ingress of water and steam during tray washing and sterilisation processes. In the embodiments illustrated in FIG. 2, the side walls, end walls and base are each of a sheet of metal. The side and end walls are dimensioned and arranged so that the tray has a generally rectangular form. In other embodiments, the tray may have a generally square form in which the side and end walls have similar lengths.

The tray may have a height of approximately 90 mm, a width of approximately 250 mm and a length of approximately 500 mm. In other embodiments, the tray may have a height of approximately 70 mm. Also, in other embodiments, the tray may be approximately square and have a side length of approximately 250 mm.

In FIG. 2, the base 210 is riveted to the side walls. In other embodiments, the base may be removable from the tray. For example, a lower portion of the side walls and one end wall may include a slot into which the base may be slidingly received. The other end of the tray may include a clip, lock or other fastener by which a user can selectively secure the base within the tray or remove the base from the tray. This may be beneficial to improve the cleanability of the tray and/or the visibility of instruments within the tray, as described in greater detail below.

Additionally or alternatively, the base of the tray may be in the form of a mesh rather than a perforated sheet. The base of the tray may have a border and a plurality of wires extending in criss-cross fashion and attached to the border so as to define a mesh. The apertures in the mesh defined by the spacing of the wires should be less than the smallest dimension of the smallest item to be stored in the tray in order to avoid the item falling through the base. The provision of a mesh base either permanently attached to the remainder of the tray or removable from the tray may also provide improvements in cleaning and/or visibility of items within the tray as described in greater detail below.

Either or both ends of the tray may include a recess 218 into which a label or nameplate may be releasably attached to indicate the contents of the tray. The label or nameplate may indicate the surgical procedure with which the contents of the tray are associated and/or the specific contents of the tray, for example in terms of the particular stage of a surgical procedure or the intended use of the contents of the tray during the surgical procedure. Additionally or alternatively, the label or nameplate may include the name of the manufacturer and/or the surgical kit name of the intended contents of the surgical instrument tray.

As illustrated in FIG. 2, the tray 200 does not bear any markings or other indicia indicating the surgical procedure and/or instrumentation that it is to be used with. Further, the tray 200 does not include any content specific formations limiting its intended use. Hence, tray 200 is a generic tray which can be used to hold instruments for any surgical procedure and/or stage of surgical procedure. It is only subsequently, as described in greater detail below, that the generic tray 200 is customised to form a surgical instrument tray 100 adapted to hold a particular combination of surgical instruments and/or parts of surgical instruments.

As illustrated in FIG. 1, the generic tray 200 is used with an insert 300 which is adapted to hold the surgical instruments and/or parts of surgical instruments and also to identify the surgical instruments and/or parts of surgical instruments. For the sake of brevity, the expression surgical instruments will generally be used below to refer both to individual instruments and also parts or components which are assembled to form a surgical instrument. In some parts below, the distinction between the parts of a surgical instrument and surgical instrument will be made explicit for clarity.

FIG. 3 shows a perspective view of an outlines part 310 of the insert 300. The outlines part 310 has a generally skeletal or framework construction including a plurality of parts generally having the shape of the outline of a corresponding surgical instrument or part of a surgical instrument and are connected together by other pieces of material to give the overall part 310 its form. The outlines parts 310 does not need to be constructed from separate outline parts and connecting parts which are then attached together. Rather, the outlines parts 310 may be moulded, from a suitable plastic or polymer. In other embodiments, the outline part 310 may be formed from a single sheet of metal which is then cut to remove material and then folded to give the outline part 310 its eventual form. As explained in greater detail below, the outline parts are highlighted having a different, highly contrasting colour, to the other parts of the outlines part 310.

Purely by way of example, the outlines part 310 includes eleven separate outlines. A first outline 312 has a shape corresponding to the outlying shape of a first part of an offset reamer driver instrument, a second outline 314 has the shape of the outline of a second part of an offset reamer driver instrument, a third outline 316 has the shape of the outline of a third part of an offset reamer driver instrument and a fourth outline 318 has a shape corresponding to the outline of a fourth part of an offset reamer driver instrument. The four separate parts of the offset reamer driver instrument are assembled to form the offset reamer driver instrument in use. However, they are stored separately in disassembled state in the tray to facilitate cleaning and sterilization of the parts of the reusable instrument.

A fifth outline 320 has the shape of the outline of a first part of an offset cup impactor and a sixth outline 322 has the shape of the outline of a second part of the same offset cup impactor.

Seventh to eleventh outlines 324, 426, 328, 330 and 332 have a shape corresponding to the outline of first to fifth liner impactor tips which may be used with the cup impactor instrument. As illustrated, the seventh to eleventh outlines are generally in the form of a circular annulus as also illustrated, in respect of seventh outline 324, the outline part 324 has a generally highly contrasting colour compared to the adjacent part of the network or framework connecting the outline parts. In the illustrated embodiment, the outline is white whereas the surrounding and/or remaining parts of the insert are black. However, this is not shown for the remainder of the outline for the sake of clarity. However, it is illustrated in FIGS. 7 and 8 below. Alternatively, the outlines may be black and the surrounding and/or remaining parts of the insert are white. Other combinations of highly contrasting colours may also be used.

The contrasting colours of the outlines and connecting parts may be realised by overmoulding, printing, laser marking or anodised coating, depending on the specific construction and/or the material of the outlines part 310. The outlines part may also be created by 3d printing of metal or plastic or a combination thereof in different colours.

Each of the first to firth outlines has at least one or more supports associated with it to allow the corresponding surgical instrument or part of a surgical instrument to be held above and in registration with its corresponding outline. As illustrated in relation to the second outline 318, a land 340 extending between parts of the outline 318 defines a first central aperture 342. Portions of land to either side of the central land 340 each define first and second side apertures 344, 346. These apertures provide a female attachment formation by which a support may be attached to the outlines part 310 as described in greater detail below and in particular with reference to FIGS. 4 to 6.

The outlines part 310 includes first and second side walls, e.g. first side wall 350 and first and second end walls, e.g. end wall 352. As schematically illustrated in FIG. 3, the side and end walls also define a plurality of apertures therein, e.g. aperture 354, again to allow for the ingress of water and steam during tray washing and sterilization procedures respectively.

As explained above, four of the eleven outlines correspond to four separate parts of a single surgical instrument which are assembled together in use and which are disassembled for storage and/or washing of the instrument. The four outlines corresponding to the four parts of the surgical instrument are grouped together in the outlines part 310 to aid the user in identifying that those parts are to be assembled in to a single instrument and may also be further configured to indicate to the user how the parts should be assembled and also the sequence of assembly and/or disassembly. This is described in greater detail below.

The side and end walls of the outlines part 310, when located in the tray, suspends the upper surface and the outlines above and spaced away from the base 210 of the tray. As discussed in greater detail below, it is believed that this helps to increase the visibility of the outlines and/or surgical instruments within the tray.

A tab 360 may also be provided toward a first end of the outlines part. The tab 360 is illustrated in FIG. 3 in a folded configuration. However, the tab may be folded upwardly so as to extend substantially perpendicularly to the plane of the upper surface of the outlines part 310 so as to provide a surface for bearing information indicating the surgical procedure with which the tray is to be associated and/or the in the surgical instrument in the tray, as described in greater detail below.

FIG. 4 shows a perspective view of the insert 300 comprising the outlines part 310 and a plurality of supports attached to the outlines. For example, first and second supports 370, 372, are attached to the first outline 312. Third and fourth supports 374, 376 are attached to the second outline, 318. Supports 370 and 372 are the same and supports 374 and 376 are the same. The first and second supports are similar to the third and fourth supports in that they generally have a pair of limbs defining a concavity within which a part of a surgical instrument may be received in use. Fifth support 378 also acts generally as a clip, similarly to the other supports. However, support 378 has a generally different configuration comprising a pair of limbs which are arranged to be received within an aperture of a surgical instrument so as to releasably hold the surgical instrument in place.

Outline 320 has associated with it the fifth support 378, a sixth support 380 and a seventh support 382. The construction of the sixth support 380 is illustrated in greater detail in FIG. 5. As illustrated in FIG. 5, each of the supports, other than support 378, generally comprises a pair of limbs 382, 384, extending upwardly from a body 386 and defining a generally circular concavity 388 between them. The free end of each limb includes a respective protrusion e.g. 390. An inner surface of the support 392 bares first, second and third ribs 394, 396, 398 which stand proud of the inner support surface 392. The ribs 394, 396, 398 act as spacers to hold the inner surface 392 of the support away from the outer surface of the corresponding part of the instrument received therein so that there is a space between the outer surface of the instrument and the support other than the places where the ribs engage the outer surface of the instrument. This helps to prevent the supports from sealing against the outer surface of the instrument in use so that the outer surface of the instrument is exposed during washing and the support does not seal against the outer surface of the instrument to prevent that part from being properly washed.

A central male member 400 and a first side male member 402 and a second male side member 404 extend from a lower part of the body 386 of the support 380. The first, second and third male members 400, 402, 404 provide a male attachment mechanism for attaching the support to corresponding apertures defined by material adjacent the outline.

The support 380 is made from a resiliently deformable material. For example, the support may be made from a natural or synthetic rubber and in particular a polymer, such as silicone. Other suitable materials include PTFE, overmoulded stainless steel (e.g. overmoulded with silicone) or plastic coated stainless steel (e.g. coated with nylon). The outer surface of the supports is preferably non-metallic to reduce or avoid any damage to the instruments or arts held therein. In some embodiments, the supports may be entirely non-metallic. The resilience of the material from which the support is made provides a sprung action such that the support 380 can act as a clip to releasably receive the surgical instrument. Also, the deformable nature of the material allows the male members to be inserted into and received in the corresponding apertures in the rigid material of the outline.

FIG. 6 shows a partial section through support 380 when mounted on outline 320. The central male member 400 is received within a central aperture. A pair of ribs 401, 403 extend from side faces of the central male member. Each rib cooperates with the corresponding side male member to define a recess within which a portion of the outline is captured when the outer male member is received in an outer aperture. Capture of the outer male members 402, 404 in respective outer apertures and receipt of the parts of the outline within the respective recesses acts to increase the rigidity of the body part of the support. Hence, the attachment formation both allows the support to be securely attached to the outline and also acts to increase the rigidity of the support to increase its spring force thereby allowing a more flexible material to be used, to aid attachment, while also providing a sufficiently strong spring force to securely hold the instrument in place. As noted above, the ribs 392, 394, 396 help to prevent the support member from otherwise binding or sealing against the instrument to prevent cleaning.

Further, the central male members 400 for each support may extend downwardly from the outlines so that the free end 406 of the central male member engages with the upper surface of the base of the tray. Hence, the central male member may also be used to help support the outlines above the base of the tray. This can be particularly beneficial if the outlines part is more delicate, for example owing to its networked and/or skeletal constructions and/or its material. In some embodiments only a sub-set of the supports include a central male member dimensioned to extend down to the upper surface of the base of the tray so that only certain parts of the outlines are more robustly supported. Generally speaking, the central male member has a length corresponding to the thickness of the outlines part and the height 408 by which the underside of the outlines is suspended above the upper surface of the base of the tray.

FIG. 7 shows a perspective view of the insert 300 separate from the tray 200. FIG. 8 shows a perspective view from above of the insert 300 located within the tray 200 and occupying the inner space defined by the walls of the tray. As best illustrated in FIG. 8, the outlines of a first outline colour and the parts of the insert other than the outlines are in a second insert colour and there is significant contrast between the two. For example, in the illustrated embodiment, the outlines are white and the remainder of the insert is black. Preferably, the parts of the outline part providing the framework and/or connections between the outlines and the supports are the same. This helps to improve the visibility of the outlines within the tray.

As also illustrated in FIG. 8, each outline may have a land associated with it, e.g. land bearing markings, such as alpha numeric characters, indicating the surgical instrument corresponding to the outline. For example, gland 420 bares a number corresponding to a surgical instrument identification number or the part of the surgical instrument corresponding to outline 322. Additionally or alternatively, a further marking may be provided either on a gland or as part of the outline, indicating the surgical instrument with which the outline is associated. For example, outline 322 includes, in reverse characters, the wording “offset cup impactor” indicating that the part associated with outline 322 is a part of an offset cup impactor instrument.

As can also be understood from FIGS. 7 and 8, the sidewalls of the outline part 310 separates the outlines away from the actual base 210 of the tray. This helps to improve the visibility of the outline when viewed against the background of the base of the tray which is visually noisy owing to the plurality of perforations therein.

It is further believed that raising the outlines above the base of the tray improves the visibility of the outlines. If the outlines are provided on or flush with the base of the tray, then the outlines are only all clearly visible when viewed substantially directly from above. By raising the outlines away from the base of the tray and toward the mouth of the tray, the angle at which all of the outlines are visible is increased so that the outlines do not need to be viewed from substantially directly above in order to be clearly viewed. This may be of benefit to human users of the tray and also to machine vision systems associated with robotic or computer assisted surgery systems and/or tray loading systems so as to more easily be able view the outlines within the tray.

It can further be seen from FIG. 8 that each outline, e.g. outline 322, is in the form of a strip. In the illustrated embodiment, the strip has a width of approximately 4 mm. However, other outline widths within the range of approximately 3 mm to 6 mm are also useful. If an outline is too narrow, then it is harder to distinguish for a viewer. However, if the width of the outline is increased too much, then parts of the outline may merge with each other and the outlines no longer function as an outline. Further, they can present larger surface areas to be cleaned. Therefore, there is an optimal range of widths which competing requirements of acting as an outline while also being easily visible. Also, for embodiments in which the outlines part is made of or includes metal, the greater the width of the outline, the greater the mass of material and therefore the greater the ability for the outline to dry after cleaning owing to the greater thermal mass present.

Each strip of material of each outline may define at least one aperture or void within the outline.

As can be seen in the Figures, the insert is provided as an entirely separate part to the tray 200 and as a self-contained or independent unit. Hence, the insert 300 can be freely removed from the tray without requiring any disassembly or deconstruction of the tray and/or insert. In some embodiments one or more releasable fasteners, such as a bolt, screw, catch or clip, may be used to retain the insert within the tray. However, the insert can still easily be removed from the tray, by releasing any releasable fasteners, and without requiring any disassembly of the tray and/or insert.

In a further embodiment of the surgical instrument tray, rather than providing a single insert, a plurality of separate inserts may be provided and which are configured to be received within the interior space of the tray. Preferably, the plurality of inserts tile the base of the tray so that there is no wasted space. For example, a first insert may be square and a second insert may be rectangular, or a first insert and a second insert may each be square or may each be rectangular, so that the first insert and second insert can be received within the rectangular tray. For further example, a first insert and a second insert may each be rectangular, or each be J, or L, shaped so that the first insert and second insert can be received within the rectangular tray. Other combinations of and types of insert shapes may also be used. Further, the number of inserts may also be greater than two. For example, the surgical instrument tray may include two, three or four separate and/or different inserts in some embodiments.

Each of the plurality of inserts will include its own plurality of outlines and corresponding supports and so each insert may be generally similar in construction to the insert illustrated in FIGS. 3 to 8 and described above.

The provision of a plurality of inserts may have a number of benefits. For example, a plurality of inserts may enable flexible surgical instrument kitting generally and also smart kitting flexibility. In some instances, there may be a need for a surgical instrument tray to have a first insert with one or more core surgical instruments, such as the off-set reamer driver, and a second insert with one or more patient specific surgical instruments. Additionally or alternatively, non-standard surgical procedures and/or patient specific surgical procedures may more easily be supported by allowing a particular surgical instrument kit to be customised by providing a first insert with one or more core surgical instruments and a second insert with one or more surgical instruments not typically sued or provided, but which may be appropriate for a customised or otherwise non-standard surgical procedure or workflow depending on, for example, the patient and/or surgeon and/or implant.

FIG. 9 shows a perspective view of a further embodiment of the tray 220 and insert 450 of the invention. In FIG. 9, only the outline parts of the insert 450 are shown and not also the side walls and supports, simply for ease of illustration. The second embodiment of the tray 220 is generally similar to the first embodiment 200 other than there being an aperture 222 defined in an end wall 224 of the tray. Further, the second embodiment of the insert 450 is generally similar to the first embodiment 300. However, the second embodiment of the insert 450 includes a tab of material 452 standing generally upright relative to the plane of the upper surface of the insert. Tab 452 is positioned and sized such that when the insert 450 is received within the interior space of the tray, then the outer surface 454 of the pad 452 is in registration with aperture 222 and therefore is visible to a user. The outer surface 454 may bear markings and/or indicia presenting information to a user. The information may include an indication of the surgical procedure with which contents of the tray are to be used and/or an indication of the surgical instruments and/or parts of surgical instruments contained in the tray. Additionally or alternatively, the outer surface 454 may bear markings and/or indicia indicating a surgical instrument identification number. In some embodiments, the information may include any one, any plurality of, or any combination, of: a brand; an implant product family name; an instrument kit variant; a barcode; a 2D barcode; and/or a product code.

Hence, the tray 220 can still be provided as a generic tray without any markings or indicia indicating its intended use. However, the information specifying the use for which it has been customised by the insert is still visible to the user without having to open the tray.

FIG. 10 shows a plan view of a third embodiment of an outlines part 500 located within the interior of a further embodiment of a tray 510. Similarly to the previous embodiments of the outlines part, the outlines part 500 includes first to fourth outlines 312, 314, 316, 318 corresponding to four different parts of a first surgical instrument and fifth 320, sixth 322, seventh 324, eighth 326 and ninth 328 outlines corresponding to first to fifth parts of a second surgical instrument.

The outlines part 500 similarly include at least one support e.g. 502, associated with each outline to releasably hold the corresponding surgical instrument part above and in registration with its corresponding outline. Similarly to the first and second embodiments, the outlines part 500 is in the form of a framework or skeletal formation in which the outlines are connected to each other by connecting portions.

The second embodiment of the tray 510 is generally similar to the first embodiment except that the side walls include shoulders defining a plurality of slots, e.g. slot 512, and that a plurality of beams e.g. beam 514, are located in opposed pairs of slots at various positions along the longitudinal length of the tray 510. The lower part of the supports extends downwardly and engage with the beams and are securely fastened thereto, for example, by a screw or similar, passing through the beams which are perforated, similarly to the base of the tray. The perforations provide ingress of steam for sterilization, ingress and drainage of water during cleaning, and also optionally a locating mechanism for fasteners and the like.

The third embodiment of the outlines 500 further illustrates how the outlines can be configured, in a variety of ways, to indicate to the user that some of the parts of the surgical instruments within the tray are intended to be assembled together to form a surgical instrument.

For example, in FIG. 10, the four outlines 312, 314, 316, 318 corresponding to the four parts of a first surgical instrument, e.g. an offset reamer driver, are grouped together in a first region, whereas the first to fifth outlines 320 to 328, corresponding to first to fifth parts of a second different instrument, e.g. an offset cup impactor, are also grouped together but in a second separate region. Hence, grouping of the outlines is a first way in which the outlines may be configured to indicate that the surgical instrument parts are intended to be assembled into a specific surgical instrument.

Additionally or alternatively, colour coding may be used to configure the plurality of outlines to indicate which ones are associated with parts of a surgical instrument to be assembled together. For example, the first to fourth outlines may have a first colour and the fifth to ninth outlines may have a second different colour. For example, the first to fourth outlines may be coloured white and the fifth to ninth outlines may be coloured yellow. Furthermore, colour coding may also be used to indicate the sequence in which the parts are assembled. For example, if the first to fourth outlines were coloured blue, then the first to fourth outlines may be coloured with different shades of blue. For example, the second outline 314 may be coloured a lightest shade of blue, then the third outline 316 with a slightly darker shade of blue, then the first outline 312 with a darker shade of blue, and then the fourth outline 318 with the darkest shade of blue. The increment in shading then indicates the sequence with which the parts are to be assembled wherein the first instrument part, corresponding to outline 314, is initially assembled with the second instrument part, corresponding to outline 316, and then that construct assembled with the third instrument part, corresponding to outline 312, before being assembled with the fourth instrument part, corresponding to outline 318, in order to assemble the instrument.

Similarly, if the fifth 320 and sixth 322 outlines, and the seventh 324 to ninth 328 outlines, were coloured red, then different shades or intensities of red may be used to indicate the sequence of assembly. For example, the fifth outline 320 may be a lighter shade of red, the sixth outline 322 may be a darker shade of red and each of the seventh to ninth outlines 324-328 may all be a same darkest shade of red to indicate that the surgical instrument part corresponding to the fifth outline 320 is first assembled with the surgical instrument part corresponding to the sixth outline 322, and then a part from a one of the seventh 324 to ninth 328 outlines, in order to assemble the instrument.

Disassembly of the instruments is simply the reverse, in which case the parts corresponding to the darker outlines are disassembled and returned to the tray initially and then the parts corresponding to the lighter outlines. The reverse order of shading may also be used to indicate the sequence of assembly/disassembly, i.e. darkest to lightest for assembly and lightest to darkest for disassembly.

Additionally and/or alternatively, other indicia may be used to configure the outlines to indicate that the parts are intended to be assembled together and/or the sequence with which the parts should be assembled and/or disassembled. For example, as illustrated in FIG. 10, the connecting parts between the outlines may have features, in particular apertures, in the shape of arrows or triangles for the connecting parts extending between outlines of the parts intended to be assembled into the surgical instrument. Hence, the connecting parts between the first, second, third and fourth outlines include arrows showing the corresponding parts are intended to be assembled together. In contrast, a connecting part between parts of surgical instruments not intended to be collected together may bear different indicia. For example, connecting part 520 includes perforations in the form of circular holes which indicates that the parts are not intended to be assembled together. Further the perforations also permits the ingress of water and team during cleaning and sterilization. On the other hand, the connecting parts 522, 524 between the fifth 320 and sixth 322 outlines includes arrows showing that those parts are intended to be assembled together. Indeed, the arrows, in pointing from the outline 322 for the second part to the outline 320 for the first part of the second instrument, also indicates the sequence of assembly by indicating that the second part is to be assembled with the first part.

Further indicia may be provided on the connecting parts, and/or colour coding, in order to indicate the sequence with which the parts of the instrument are intended to be assembled.

Further, the relative positioning of the outlines may be a way in which the outlines are configured in order to indicate the sequence in which the parts are intended to be assembled and/or to correspond to the surgical workflow. For example, starting toward the left hand side of the tray, when viewing FIG. 10 in landscape format, and reading left to right, the first outline for the second instrument parts encountered is outline 320, the next outline moving progressively right is outlain 322 for the second part, and, continuing left to right, the final outlines encountered are 324, 326, 328, each of which corresponds to a final part of the second instrument. Hence, the relative positioning of the outlines may alternatively and/or additionally be used to indicate the sequence in which they corresponding parts are intended to be assembled and/or disassembled. However, this may not always be possible, for example in circumstances in which surgical instruments and/or parts need to be arranged depending on their shape and/or size.

FIG. 11 shows a plan view of a fourth embodiment of the outlines part 530 and using the same second embodiment of the tray 510 as shown in FIG. 10. In this fourth embodiment of outlines part 530, the outlines part is configured to indicate that the corresponding parts of the surgical instrument are intended to be assembled together and also the sequence with which the various parts are intended to be assembled into the instrument. Each outline includes a land or tab, e.g. tab 532, bearing alpha numeric characters. In the illustrated embodiment, the alphas are the same for the parts of the same instrument and the numeric element indicates the sequence with which the parts are assembled and/or disassembled. Hence, the first to fourth outlines 312, 314, 316, 318 are associated with indicia A3, A1, A2 and A4 respectively. The “A” indicates that these four outlines correspond to parts of the same surgical instrument and the 1, 2, 3, 4 indicates the sequence in which the parts are assembled and/or disassembled.

For example, during assembly, a first part corresponding to outline 314 and labelled A1 may be removed first, then inserted into a part a corresponding to outline 316 and labelled A2. Then a third part corresponding to outline 312 labelled A3 inserted into the assembly of the first and second parts, before a fourth part corresponding to outline 318 labelled A4 is used to complete the instrument assembly.

The sequence of disassembly is simply the reverse with the fourth instrument part corresponding to outline 318 labelled A4 being removed first and placed in the tray, before the third instrument part corresponding to outline 312 labelled A3, then the second instrument part corresponding to outlain 316 and labelled A2, and finally the first instrument part corresponding to outline 314 labelled A1.

Similarly, the fifth to ninth outlines 320, 322, 324, 326, 328, also have a land or tab associated therewith, e.g. land 534. Each land similarly bears alpha numeric characters but differing to those associated with the first group of outlines. Hence, the fifth to and sixth outlines are designated B1 and B2 and the seventh to ninth outlines collectively as B3. The character “B” indicates that these parts are parts of the same surgical instrument, and a different surgical instrument to the parts corresponding o the first to fourth outlines. The numeric values 1, 2, 3 again indicate the sequence of assembly and/or disassembly of these parts into the second surgical instrument. For example, the first and second parts may be separate parts an offset cup impactor instrument and the third parts may be different tips for the impactor instrument.

The assembly and/or disassembly sequence indicating features may correspond to a surgical technique document describing the assembly and/or disassembly of the instrument to the user.

Again, colour coding may be used additionally or alternatively in a similar manner to as described above.

Various methods in which the surgical instrument tray may be used or using the surgical instrument try will now be described.

A particular benefit of the surgical instrument tray is that the tray is generic and may not include any markings or other features which might limit its use to a particular surgical procedure or instrument. Rather, the intended use, in terms of its contents, is determined by the outlines which serve both to indicate the intended contents of the tray and also can assist with correctly placing the surgical instrument and/or parts of surgical instruments within the tray.

FIG. 12 shows a flow chart illustrating a method of producing one or more surgical instrument trays including the appropriate surgical instruments and/or parts of surgical instruments. The method of producing the surgical instrument tray or trays 600 may begin at 602 at which a particular surgical procedure is selected, for example a total knee replacement surgical procedure or a total hip replacement surgical procedure. However, as discussed above, the surgical instrument tray is not limited to orthopaedic surgical procedures and can be applied to any surgical procedures in which multiple surgical instruments are/or surgical instruments made from multiple parts may be used.

The selected surgical procedure may have a plurality of specific surgical instruments and/or kits of surgical instruments which may typically be required by a surgeon. A list of the required surgical instrument trays may be available. At 604, a generic tray is obtained. As noted above, the tray itself is generic and therefore multiple trays may be manufactured without needing to know their intended future use. At 606, the plurality of outlines for the surgical instruments and/or parts of surgical instruments intended to be placed within the tray is identified and placed within the interior space of the tray. In embodiments using the insert part, this would merely involve placing the insert part within the interior space of the tray, and potentially operating any clip, or fastener mechanism, to temporarily retain the insert within the interior space of the tray. However, the clip or fastener is not a permanent fastener and is easily operable to allow inserts to subsequently be removed from the tray in case there is a change to the layout of the surgical instruments to be received in the tray and/or the design of any one or more of the surgical instruments and/or to use the generic tray for another group of surgical instruments or indeed another surgical procedure entirely. Hence, the ability to easily add and remove the insert from the generic tray improves the efficiency with which inserted instrument trays may be reconfigured to accommodate changes.

Hence, at 608, the insert bearing the plurality of outlines is placed in the generic tray. A list of the surgical instruments and/or parts of the surgical instrument to be loaded into the tray may be available to a user. Irrespective of the availability of a list of surgical instruments and/or parts to be loaded into the tray, at 610, the outlines may be viewed to help both identify and/or confirm the surgical instrument and/or part of the surgical instrument to be placed in the tray and also the location within the tray where each surgical instrument and/or part is to be placed. Hence at 612 using the outlines, a surgical instrument or surgical instrument part is identified and selected from those available for loading into the tray. At 614, the surgical instrument or part may then be placed in the support or supports above and in registration with the outline.

Preferably, the outlines have an outer perimeter and the dimensions of the outer perimeter of the outlines are slightly greater than the corresponding dimensions of the surgical instrument or part. Hence, when the surgical instrument or part is placed in registration above its corresponding outline, at least a small portion of the outer perimeter of the outline is preferably still visible. This can provide a verification mechanism in that if the outline is entirely obscured, this may indicate that a similar but wrong surgical instrument or part has been placed in the wrong location. Alternatively, if there are overlaps or no overlap with certain parts of the outline by the surgical instrument or part, then this may also indicate that the wrong or very similarly shaped surgical instrument or part has been placed in the tray. However, if there is a generally constant thickness of outline visible around the perimeter of the surgical instrument or part when located in the supports, e.g. approximately 1 mm around they periphery of the surgical instrument or part, then this provides further confirmation that the correct surgical instrument or part has been selected and placed at the correct location within the tray. If it is determined that the wrong surgical instrument or part has been placed, then the current surgical instrument or part is removed and the outlines can be viewed again at 610 and the process repeated. Assuming that the correct surgical instrument or part has been placed in the tray at 614, then at 616, it can be determined whether loading of the tray has been completed. Again, this can involve inspecting the outlines to see if any outline is still clearly visible, based on the contrasting colour between the outline and surrounding parts. Hence, the visibility of the entirety of an outline can also be used to check whether loading of the tray has been completed or not.

As discussed above, it is believed that raising the outlines above the base of the tray improves the ease with which the outlines may be visible. If the outlines were positioned at the base of the tray, then those toward the edges of the base may not be visible unless viewed from substantially directly above. However, by raising the outlines above the base of the tray, outlines towards the periphery may be more easily visible without being viewed directly from above. Hence, providing the outlines on a separate part to the base both allows the generic tray to be provided as a generic part and can also help to improve the visibility of the outlines by suspending them above and spaced away from the base.

If at 616 it is determined that the tray is not complete, then, as illustrated by process flow line 618, the method returns to 610 and the outlines can further be viewed to help determine which surgical instrument and/or parts are still required to be loaded and the process repeats. Hence, the method is repeated until it is determined at 616 that the tray is complete, i.e. that all of the surgical instruments and/or surgical instrument parts intended to be provided in the tray have been placed in their corresponding supports. It will be appreciated that in some instances, not all the items which may be placed in the tray would need to be placed in the tray in order for the tray to be considered complete.

In some embodiments, one or more plates having generally the same outline shape as the corresponding outline may be provided and which can be received in the corresponding support so as to cover the outline. This may be done to provide a visual indication that a surgical instrument or part has purposefully been omitted from the tray.

Once the current tray is determined to be complete at 616, then at 620, it is determined whether all of the trays currently required have been completed or not. For example, in some embodiments, the same group of surgical instruments and/or parts may need to be loaded into trays in order to provide a plurality of those particular kits of instruments and/or parts. Additionally or alternatively, all of the trays required for a particular surgical procedure may be produced. For example, a local warehouse may build ten trays each with the same particular layout to hold on standby for a set of hospitals. For further example, a local hospital may build a set of trays for a particular surgery or a particular day of surgeries or a week of surgeries. Hence, if it is determined that further trays are to be produced at 620, then the method returns, as illustrated by a process flow line 622 to and another generic tray is obtained, then at 606, the appropriate insert is selected in order to customise the surgical instrument tray for its intended contents. Hence, the method repeats generally as described above until it is determined at 620 that all of the trays to be produced have been produced. The method may then end.

The method 600 described above may be carried out by one or more persons or an automated tray loading system, for example using vision system and robotics, or a combination of the two in a semi-automated procedure. It is believed that the use of outlines and in particular the contrast between the colour of the outlines and surrounding parts may be a particular benefit for automated systems using machine vision and robotics as enhancing the ability to correctly select and/or position and/or verify the surgical instruments or parts to be loaded into the tray.

FIG. 13 shows a flow chart illustrating a method of use of the surgical instrument trays 700, and including various methods according to various aspects of the invention. The invention is not limited to the overall combination of steps and various different methods involving different combinations of the individual steps may also give rise to various inventive methods.

At 702, the surgical instrument trays required for a particular surgical procedure are identified and retrieved from storage. The surgical instrument trays are then moved to the operating room or theatre at 704 and at 706 any external wrapping or sterilization containers as typically used to maintain the sterility of the trays may be removed. After removing the wrapping, the lid of the tray is removed after placing the tray where required.

Optionally, in embodiments in which the base is removable, at 708, the base of one or more of the trays may be removed. By removing the base of the tray, a constant visual background may be provided so as to improve the visibility of the outlines based on their contrast with a uniform background. For example, if the base is in the form of a metal sheet with plurality of perforations, the perforations are required in order to allow cleaning of the tray. However, the perforations in the base give rise to a visually noisy background which means that the profiles may be harder to discern. Hence, by removing the base, the visual background will likely be either a solid silver colour, for example from a surgical table, or a surgical drape colour, for example blue, providing a uniform visual background. This can increase the ability to differentiate between the background and the outlines. It is believed that the use of a mesh can also improve the visual contrast as there is less solid material in the base and a greater proportion of the base is voids between the wires forming the mesh. Hence, a non-removable mesh may provide some compromise between improved cleanability and the ability of the outlines, compared to a perforated solid base. However, in other embodiments, a mesh base may also be removable.

Alternatively, at 708, and also optionally, the insert may be removed from the tray while holding the surgical instruments. This can provide similar benefits in improving the visibility of the outlines to removing the base.

At 710, the surgical instruments and parts within the tray, or on the insert if the insert has been removed from the tray, may be viewed, for example by the surgeon, scrub nurse or other person in the operating theatre. When and as required at 712, the surgical instruments and/or parts may be removed from the surgical instrument tray or insert.

If at 712 a surgical instrument provided in whole form in the tray is required, then the surgical instrument may simply be removed from the tray/insert. The high contrast outline may assist in identifying which particular instrument from the tray is required.

If a surgical instrument which requires assembly is needed, then the user may view the outlines to help identify both the parts within the surgical instrument tray which are assembled to form the instrument and also to identify the sequence in which the parts are to be assembled. As discussed above, the outlines may be configured in a number of ways in order to impart this information. For example, the outlines may be grouped together, colour coded, include indicia or markings presenting information indicating the parts are parts of the same instrument and the sequence of their assembly, and/or symbols, and/or the relative positions of the outlines within the tray and/or combinations of these. Hence at 712, the user may identify the parts of a surgical instrument and the sequence of their assembly and remove the parts of the surgical instrument from the tray in the appropriate sequence to assist assembly. This may improve the efficiency with which an experienced user can assemble a surgical instrument and/or assist a less experienced user in being able to assembly a surgical instrument and/or assembly of a surgical instrument which is uncommonly used.

At 714, when a surgical instrument has been finished with and is no longer required, then the user may view the outlines in the tray to help assist correctly disassemble and/or replace the surgical instrument within the tray or insert. If a multi-part surgical instrument is no longer required, then the user may disassemble the surgical instrument and sequentially place the parts of the surgical instrument in the corresponding supports of the corresponding outlines. The information presented by the outlines in the tray may assist the user in determining the sequence of disassembly, being the reverse of the sequence of assembly. Hence, by viewing the outlines in the tray, the user may be assisted in determined how to disassemble the surgical instrument. However, in some circumstances, users may not replace surgical instruments within trays during the operating theatre and may simply place the used surgical instruments in a bin or other receptacle for subsequent disassembly and cleaning. Hence, step 714 may be optional.

At 716 it is determined whether the tray or trays are still currently required. If the tray or trays are still required, then the method returns to step 710, as indicated by process flow line 718 and the user can continue to view the instruments in the tray or trays in order to identify and remove the instruments and assist in assembly of the surgical instruments for multiple parts.

Once a particular tray is no longer required and/or at the end of the surgical procedure, optionally, at 720, a user may check the contents of the tray. If the surgical instruments or parts of surgical instruments have been replaced in the tray during the surgical procedure, then optionally at 720, a user may view the outlines within the tray to help establish that the instruments and/or parts have been correctly placed in the correct tray and/or that the tray has been completely repacked, for example by the presence of an outline with no corresponding instrument or part.

Again, optionally at 722, if the base has previously been removed, then the base of the tray can be replaced. Alternatively, optionally at 722, if the insert was previously removed from the tray, then the insert be replaced back in the tray. The lid may then be replaced and then the tray or trays removed from the operating theatre and taken to a cleaning facility.

At 726, at the cleaning facility, if the instruments and/or parts have not been provided within their original tray, but rather have been provided separately, for example in a bin or other receptacle, then the surgical instruments can be returned to their corresponding positions within the tray, based on the outlines. Any surgical instruments which need disassembly can be disassembled using the outlines as guidance as to the sequence with which the parts should be disassembled and placed in the supports above their corresponding outlines in the tray. Optionally, in embodiments in which the base is removable, at 728, the base of the tray may be removed. The base and lid may be washed separately to the tray and instruments. At 730, irrespective of whether the base has been removed or not, the contents of the tray may be checked by viewing the outlines to ensure that the correct surgical instruments and/or parts are present in the tray and also that all of the surgical instruments and/or parts are present in the tray as none of the outlines are unobscured. After the tray contents have been checked at 730, the tray or trays may be washed and sterilised using a conventional process.

After washing of the trays and bases and lids, the base may optionally be replaced at 734 and the lid reattached. At 736, any additional protection and/or coverings may be applied to the tray, such as wrapping as is known in the art. Once the tray or trays have been wrapped at 736, then the trays may be returned to storage at 738 for subsequent use.

Alternatively, the insert holding the instruments may be removed from the tray and the instruments and insert may be washed separately to the tray and lid. Subsequently, the insert and instruments may then be returned to the washed tray and lid and then wrapped as above.

Hence, the outlines may be used at various stages during use of the tray in the operating theatre and/or subsequently during the tray and instrument cleaning process to help assembly and/or disassembly of instruments made from multiple parts and also to guide the correct placement of instruments and/or parts within the tray and also to check completeness of the tray.

In some embodiments, the removable base may assist in visualisation of the instruments or parts or outlines with respect to a uniform background. Also, removable of the base may assist with washing and cleaning of the tray, instruments and instrument parts during the sterilisation process. Further, the position of the outlines suspended above and away from the base may help to increase the ease with which the outlines and/or instruments or parts are viewable by a user or by a machine vision system. Also, removing the insert holding the instruments from the tray may assist with visualisation and/or washing. Hence, some of the steps in the method illustrated in FIG. 13 may be carried out by automated robotic systems using machine vision systems as automated, semi-automated or supervised steps. Additionally or alternatively, some or all of the steps may be carried out by a human. Hence, in the context of the present specification, viewing should be considered to include both viewing by a human and also viewing by a machine vision system, for example using cameras or other image detection devices.

In this specification, example embodiments have been presented in terms of a selected set of details. However, a person of ordinary skill in the art would understand that many other example embodiments may be practiced which include a different selected set of these details. It is intended that the following claims cover all possible example embodiments.

Any instructions and/or flowchart steps can be executed in any order, unless a specific order is explicitly stated. Also, those skilled in the art will recognize that while one example set of instructions/method has been discussed, the material in this specification can be combined in a variety of ways to yield other examples as well, and are to be understood within a context provided by this detailed description.

While the disclosure is amenable to various modifications and alternative forms, specifics thereof have been shown by way of example in the drawings and described in detail. It should be understood, however, that other embodiments, beyond the particular embodiments described, are possible as well. All modifications, equivalents, and alternative embodiments falling within the scope of the appended claims are covered as well.

1. A surgical instrument tray for a surgical instrument having a plurality of parts from which the surgical instrument is assembled, the surgical tray comprising:
a tray having a plurality of side walls and a base which define an interior space;
a lid for closing the interior space and which is removable from the tray; and
a plurality of outlines located within the interior space, each outline having the shape of a corresponding different part of the surgical instrument and wherein each outline has at least one support arranged to receive and releasably hold the respective different parts of the surgical instrument above and in registration with the outline and wherein the plurality of outlines are configured to indicate that the different parts of the surgical instrument are assembled to form the surgical instrument and/or that the surgical instrument is disassembled into the different parts.

2. The surgical instrument tray of claim 1, wherein the plurality of outlines are further configured to indicate how the different parts of the surgical instrument are assembled to form the surgical instrument and/or how the surgical instrument is disassembled into the different parts.

3. (canceled)

4. The surgical instrument tray of claim 1, wherein the plurality of outlines are configured by being grouped together.

5. The surgical instrument tray of claim 1, wherein the plurality of outlines are configured by the relative positions of the plurality of outlines.

6. The surgical instrument tray of claim 1, wherein the plurality of outlines are configured by colour coding of the plurality of outlines.

7. The surgical instrument tray of claim 1, wherein the plurality of outlines are configured by respective indicia associated with each outline.

8. (canceled)

9. The surgical instrument tray of claim 1, wherein the plurality of outlines are connected by respective lands and each land includes an indicia that indicates how the parts are assembled or disassembled and/or the sequence.

10. The surgical instrument tray of claim 1, and further comprising a further plurality of outlines, wherein each of the further plurality of outlines has the shape of a corresponding further surgical instrument or part of a further surgical instrument and wherein each outline has at least one support arranged to receive and releasably hold the further surgical instrument or part of the further surgical instrument above and in registration with the outline.

11. The surgical instrument tray of claim 1, wherein each outline has an outer perimeter and the dimensions of the outer perimeter of the outline are greater than the dimensions of the corresponding portions of the different parts of the surgical instrument, so that the outer perimeter of the outline is visible to a user when the parts of the surgical instrument are located within the respective supports above the corresponding outlines.

12. The surgical instrument tray of claim 1, wherein the plurality of outlines are provided as part of a sheet of material which is separate to the base of the tray.

13. (canceled)

14. The surgical instrument tray of claim 1, wherein each outline is in the form of a continuous strip.

15. (canceled)

16. The surgical instrument tray of claim 1, wherein each of the plurality of outlines has an outline colour and the remainder of the surrounding material has a surround colour and wherein the outline colour and the surround colour are different colours and are contrasting colours.

17. The surgical instrument tray of claim 1, wherein the plurality of outlines are made from a sheet of metal and the plurality of outlines have been created by cutting the sheet of metal.

18. The surgical instrument tray of claim 1, wherein each support is made of a resilient, non-metallic material.

19. The surgical instrument tray of claim 1, wherein each support is in the form of a clip.

20. The surgical instrument tray of claim 1, further comprising the plurality of parts from which the surgical instrument is assembled and wherein the plurality of parts are received in and held by the respective supports above and in registration with the corresponding respective outlines.

21. A method of using a surgical instrument tray to assemble a surgical instrument form a plurality of parts of the surgical instrument, wherein the surgical instrument tray comprises a tray having a plurality of side walls and a base which define an interior space and a plurality of outlines located within the interior space, each outline having the shape of a corresponding different part of the surgical instrument and wherein each outline has at least one support receiving and releasably holding the respective different parts of the surgical instrument above and in registration with the outline and wherein the plurality of outlines are configured to indicate that the different parts of the surgical instrument are assembled to form the surgical instrument, the method comprising;
viewing the plurality of outlines to identify the parts of the surgical instrument to be assembled to form the surgical instrument;
removing the identified parts of the surgical instrument from the supports; and
assembling the identified parts of the surgical instrument to form the surgical instrument.

22. The method of claim 21, wherein the plurality of outlines are further configured to indicate the sequence in which the different parts of the surgical instrument are assembled to form the surgical instrument, and wherein the method comprises removing the identified parts and assembling the identified parts according to the sequence.

23. A method of using a surgical instrument tray to disassemble a surgical instrument formed from a plurality of parts, wherein the surgical instrument tray comprises a tray having a plurality of side walls and a base which define an interior space and a plurality of outlines located within the interior space, each outline having the shape of a corresponding different part of the surgical instrument and wherein each outline has at least one support arranged to receive and releasably hold the respective different parts of the surgical instrument above and in registration with the outline and wherein the plurality of outlines are configured to indicate that the different parts of the surgical instrument are assembled to form the surgical instrument, the method comprising;
disassembling at least one part of the surgical instrument;
viewing the plurality of outlines to identify the support for the at least one part of the surgical instrument that has been disassembled; and
placing the at least one disassembled part in the identified support above and in registration with the corresponding outline for the part.

24. The method of claim 23, further comprising:
disassembling all the parts of the surgical instrument;
viewing the plurality of outlines to identify the respective supports for all the parts of the surgical instrument that have been disassembled; and
placing all the parts in the respective identified supports above and in registration with the corresponding outlines for the parts.
